(19) United States
(12) Patent Application Publication
(10) Pub. No.: US 2025/0025240 A1
(43) Pub. Date: Jan. 23, 2025

(54) DETECTION OF A DISTAL END OF A MEDICAL INSTRUMENT

(71) Applicant: Siemens Healthineers AG, Forchheim (DE)

(72) Inventor: Alois Regensburger, Poxdorf (DE)

(21) Appl. No.: 18/753,084

(22) Filed: Jun. 25, 2024

(30) Foreign Application Priority Data
Jul. 20, 2023 (DE) 10 2023 206 888.5

(51) Int. Cl. A61B 34/20; A61B 90/00; A61M 25/01

(52) U.S. Cl. CPC A61B 34/20; A61B 2034/2065; A61B 2090/3933; A61M 2025/0166

(57) ABSTRACT

The detection of a distal end of a medical instrument in a surrounding region may be made with as little contrast medium as possible. For this purpose, a contrast medium is ejected at the distal end of the medical instrument into the surrounding region. The ejected contrast medium is determined automatically with an imaging modality in the surrounding region. The ejection of the contrast medium is then automatically reduced or terminated depending on the result of the detection.

The present patent document claims the benefit of German Patent Application No. 10 2023 206 888.5, filed Jul. 20, 2023, which is hereby incorporated by reference in its entirety.

### TECHNICAL FIELD

The present disclosure relates to a method for detecting a contrast medium in a region surrounding an outlet of a medical instrument. Furthermore, the present disclosure relates to a system with a medical instrument having an outlet for a predetermined contrast medium.

### BACKGROUND

In certain area of medicine, it is necessary to examine vessels with instruments. To do this, the respective instrument (for example catheter or guidewire) penetrates a vascular duct. Such cannulation may be performed under observation using an imaging modality. Herein, it is possible to identify whether or where the tip of the medical instrument is located in the respective vessel or how the vessel runs in the immediate vicinity of the instrument tip.

It is known that, during cannulation, a contrast medium is ejected at the tip of the medical instrument and that the contrast medium may be identified using the imaging modality. The contrast medium propagates in the respective vessel so that the course of the vessel may be visible in an X-ray image. At the same time, the tip of the medical instrument is also visible so that the physician may determine whether or where the tip of the instrument is located in the vessel or how the instrument tip may be moved further along the course of the vessel.

Bile ducts may also be examined using cannulation (e.g., endoscopic retrograde cholangiopancreatography (ERCP) cannulation). Herein, a bile duct may be penetrated with a so-called guidewire (or sphincterotome). The challenge is now to inject as little contrast medium as possible into the bile duct because the contrast medium may trigger complications such as pancreatitis.

In the past, so-called “blind cannulation” was frequently used to examine vessels if contrast medium administration was to be avoided. Herein, for example, only the guidewire is visible on an X-ray image but not, for example, the bile ducts. For less experienced users, in particular, this is associated with an increased rate of complications and with a higher proportion of patients who have to be referred to specialists due to failed cannulation.

On the other hand, cannulation is performed by experts who have a great deal of sensitivity and experience. Such experts inject a tiny amount of contrast medium at a few crucial points in time which they may just make out on the X-ray image. Physicians with little experience are strongly advised not to use this method because the method may cause medical complications if executed incorrectly.

### SUMMARY AND DESCRIPTION

Hence, the object of the present disclosure is to be able to detect contrast medium in a region surrounding an outlet or exit point of a medical instrument while minimizing the administration of contrast medium in a surrounding region.

The scope of the present disclosure is defined solely by the appended claims and is not affected to any degree by the statements within this summary. The present embodiments may obviate one or more of the drawbacks or limitations in the related art.

In one embodiment, a method is provided to detect a contrast medium in a region surrounding an exit point (e.g., at or in the region of a distal end) of a medical instrument. The method includes ejecting a contrast medium at the distal end of the medical instrument into the surrounding region, automatically detecting the ejected contrast medium with an imaging modality in the surrounding region, and automatically reducing or terminating the ejection of the contrast medium depending on the result of the detection.

The outlet or the distal end (in the present document also to be understood as the region at the distal end) of the medical instrument may therefore be detected or located. In particular, the medical instrument may be detected in its surroundings. Therefore, the medical instrument is detected in a surrounding region that surrounds the distal end. This surrounding region may be the inside of a vessel of a living being or a channel in a machine or system. The surrounding region may be limited to the inside of the vessel or the inside of the channel. This local limitation may allow conclusions to be drawn about the type of vessel or the type of channel.

In the method, the contrast medium is ejected into the surrounding region in a distal end region of the medical instrument, wherein the distal end region includes the distal end. For example, the contrast medium is guided inside the medical instrument to its distal end, where the contrast medium may escape into the surrounding region. The contrast medium may also be guided outside the medical instrument in a separate channel to the tip or distal end of the medical instrument. The contrast medium is ejected automatically, for example, with the aid of a pump that may generate a corresponding pressure or a corresponding flow rate. The contrast medium does not have to be ejected at the end face of the medical instrument but may also be ejected through an opening that is aligned perpendicular to the longitudinal direction of the medical instrument.

In a further act of the method, the ejected contrast medium is detected with an imaging modality in the surrounding region. For example, the imaging modality is an MRI apparatus or X-ray apparatus. The contrast medium may be matched to this respective imaging modality so that the contrast medium is as clearly visible as possible on the respective images. On the other hand, the contrast medium may also be compatible with the surroundings and, for example, not aggressive (for example, not injected in too large a quantity or not injected with too high a pressure or too high a flow rate). The contrast medium is detected in the surrounding region in which the distal end of the medical instrument is located. If the surrounding region is the inside of a vessel, detection is thus performed in an image region that represents the inside of the vessel. Detection takes place automatically. For example, image evaluation takes place by corresponding evaluation software. Thus, the result of the automatic detection may be a signal that represents the presence of the contrast medium in the surrounding region. A detection signal may also signal the success of an image identification task based on the contrasted images, for example that the direction or diameter of the vessel has been ascertained successfully.

Finally, in the method, the ejection of the contrast medium is automatically reduced or terminated depending on the result of the detection. For example, the detection signal is used to terminate the ejection of the contrast medium as quickly as possible. However, the contrast medium flow may also initially only be reduced depending on the detection signal. The ejection of the contrast medium may also be reduced or terminated automatically in stages. For example, only a small portion of the contrast medium may be detected in a first detection and a larger portion of the contrast medium may be detected in a second detection. Then, in the first detection, for example, the flow rate of the contrast medium may be automatically reduced, and, in the second detection, the ejection may be terminated.

In principle, the automatic reduction of the ejection of the contrast medium may also be part of a regulating process. For example, a regulating unit reduces the ejection of the contrast medium precisely so that a predetermined amount of contrast medium is detected in the surrounding region, or a predefined image identification task is solved on the basis of the contrast medium (for example, identifying the direction of the vessel). In this case, the regulating unit may also automatically increase the ejection of the contrast medium. Thus, it may be possible to provide that only a minimal amount of contrast medium is administered over a longer period of time in each case.

Advantageously, the automatic detection of the contrast medium and the corresponding automatic reduction or termination of the contrast medium administration enables the amount of ejected contrast medium to be reduced to a minimum. This means that complications may be ruled out.

The method may also be used in the context of automatic learning.

According to an advantageous development, during the ejection of the contrast medium, at least one parameter of the contrast medium is detected and controlled or regulated depending on the result of the detection. In certain examples, the at least one parameter of the contrast medium includes an amount of fluid ejected, a flow rate, a pressure, a mixing ratio, a concentration, or a combination thereof. The contrast medium flow may, therefore, be monitored in terms of its amount. For example, the contrast medium pump (hereinafter, also referred to as the pumping facility or pump) is controlled or regulated in such a way that the pump only delivers a certain volume of contrast medium. Alternatively, or additionally, the flow rate of the contrast medium may also be monitored. This may be used to draw conclusions about the amount of fluid. Alternatively, or additionally, it is furthermore possible to detect or monitor the pressure of the contrast medium. Under some circumstances, this variable may be used to draw conclusions about the amount of contrast medium if other relevant parameters in this regard are known. Other variables to be detected, such as viscosity, temperature, and the like, may also be included in the control or regulation. The mixing ratio or concentration of the injected contrast medium may be adjusted in a variable ratio with a second substance, e.g., saline solution.

In a further embodiment, on the start of the ejection of the contrast medium, a frame rate and/or radiation dose of the imaging modality is increased. The actuation signal for the pump for the contrast medium may, therefore, also be used to control or regulate the imaging modality. If, for example, the frame rate is increased, a corresponding detection may be performed more quickly or with improved time resolution in the images obtained. If, for example, the radiation dose is increased on the start of the ejection of the contrast medium, the X-ray images may have higher contrast, making detection more reliable overall. As used herein, the phrase “on the start of the ejection of the contrast medium” means that the frame rate or radiation dose may be increased before the contrast medium is detected. This enables a minimal amount of contrast medium to be detected even more reliably.

According to a further embodiment, before or at the start of the ejection of the contrast medium, a reference recording of the distal end of the medical instrument is recorded by the imaging modality and the reference recording is used to detect the contrast medium. For example, an X-ray recording of the medical instrument in use without contrast medium is obtained and stored. This reference recording may then be subtracted from a further recording obtained after contrast medium administration. This enables contrast medium propagation to be identified particularly well without disruptive structures in the surroundings.

According to a further embodiment, to reduce or terminate the ejection of the contrast medium, a pump for the contrast medium is slowed down or stopped. The result of the automatic detection may be available in the form of a detection signal. This in turn may be used to actuate or regulate the pump for the contrast medium. A corresponding interface from the detection apparatus or the imaging modality to the pump may be implemented for this purpose. In certain examples, a plurality of detection signals may be transmitted to the pump so that the pump may also be stopped in stages.

According to a further embodiment, during the detection, a propagation region of the contrast medium is detected. Therefore, not only the simple presence or absence of the contrast medium is detected during the detection, but also the propagation region or its shape or direction. For example, the propagation region may be recorded or detected in dependence on the time. The propagation region may change dynamically. This means that it is also possible to use the time component of the propagation of the contrast medium into the surrounding region to control or regulate the contrast medium ejection. Hence, if the shape of the propagation region is detected more precisely, it is possible to obtain vascular information, in particular with regard to the shape and/or identity of the vessel.

According to a further embodiment, the detection is based on image identification and the result of the detection represents whether or not a predetermined image or a predetermined image content (such as, for example, image feature or vessel feature) is identified. Automatic image identification may be used to automatically identify whether a predetermined structure is located in a recording of the imaging modality or whether further properties (diameter, direction, etc.) of this predetermined structure may be ascertained/identified by the image analysis algorithm. For example, a cloud around the distal end of the medical instrument may be specified as the image to be identified. As soon as the image analysis algorithm identifies such a cloud around the tip of the medical instrument, it may provide a corresponding detection result for the system control.

Furthermore, in one embodiment, the method may be repeated several times, wherein, on each repetition, a position of the distal end of the medical instrument is changed and thus respective contrast medium administrations into different surrounding regions and images of the respective contrast medium administrations are superimposed. This, for example, enables numerous small contrast medium administrations to take place one after the other, and a corresponding image to be recorded on each contrast medium administration. The recordings may then be superimposed so that the surrounding regions filled with contrast medium are lined up in the superimposed image. Hence, for example, the structure of a vessel may be detected with a small amount of contrast medium.

In a further embodiment, for the ejection of the contrast medium, one or more pump parameters are learned automatically in such a way that the detection takes place according to a predetermined detection result. The learning may take place using a neural network. Input parameters of the neural network may be a pump parameter and a respective detection result or an associated quality of the detection result. The output parameters may then be an optimized pump parameter. Learning the one or more pump parameters enables it to be provided that the pump only pumps precisely as much contrast medium into the surrounding region as is required for the desired detection result. For example, it is desirable for a contrast medium plume with a length of 1 cm to be visible on the image of the imaging modality. A neural network may be used to learn a respective pump parameter in such a way that, for example, on each ejection, the pump only delivers enough contrast medium to produce a contrast medium plume with a length of 1 cm.

Alternatively to the use of a neural network, it may also be ascertained after how many fractions of a second/after how much contrast medium injection, the image identification task was completed “last time.” A corresponding value may then be set for the current injection, e.g., with a small additional safety buffer. Optionally, learning may take place iteratively, for example, during a plurality of successive executions of the method. In particular, during repeated executions of the method, the pump parameter may be learned based on previous executions. In some circumstances, learning may also be patient-specific.

The object described above is also achieved by a system with a medical instrument having an outlet (in particular at or in the region of its distal end) for a predetermined contrast medium, an imaging modality for detecting a portion of the contrast medium ejected at the outlet of the medical instrument and a control facility for reducing or terminating an ejection of the contrast medium depending on the result of the detection.

The advantages and variations described above in connection with the method also apply to the system disclosed herein. Thus, the respective method acts may also be understood as functional features of respective means.

In an advantageous embodiment of the system, the medical instrument includes a catheter, a sphincterotome, or a guidewire (for example, a more complex instrument). Such instruments are predestined to examine any cavities or vessels. In each case, they have a distal end that may penetrate the respective cavity. The contrast medium may be released into the surrounding region from this distal end or end region. In particular, the sphincterotome or guidewire may represent the medical instrument of the system thus enabling examinations of bile ducts. The tip of the sphincterotome may then be reliably inserted into the desired bile ducts with minimal contrast medium administration.

In another embodiment of the system, the imaging modality includes an X-ray apparatus, an MRI apparatus, or a sonography apparatus. In principle, the imaging modality may also be based on a different type of image acquisition. The X-ray apparatus can, for example, be a C-arm, an angiography device, or the like.

According to the disclosure, a computer program or a computer program product is also provided that includes instructions, which, when executed by the aforementioned system, cause the system to perform a method as likewise described above. Hence, the computer program may represent a control program for the corresponding system, wherein the computer program may be performed in the control facility of the system and, in certain examples, with the aid of a processor and any memory means in the control facility.

For applications or situations which may arise during the method and are not explicitly described here, it may be provided that, according to the method, an error message and/or a prompt for user feedback is output and/or a default setting and/or a predetermined initial state is set.

### BRIEF DESCRIPTION OF THE DRAWINGS

FIG. 1 depicts an example of an X-ray image with a medical instrument and bile ducts.

FIG. 2 depicts an example an X-ray image with a catheter during an endovascular liver intervention.

FIG. 3 depicts a schematic representation of an embodiment of a system.

FIG. 4 depicts a schematic flow chart of an embodiment of a method.

### DETAILED DESCRIPTION

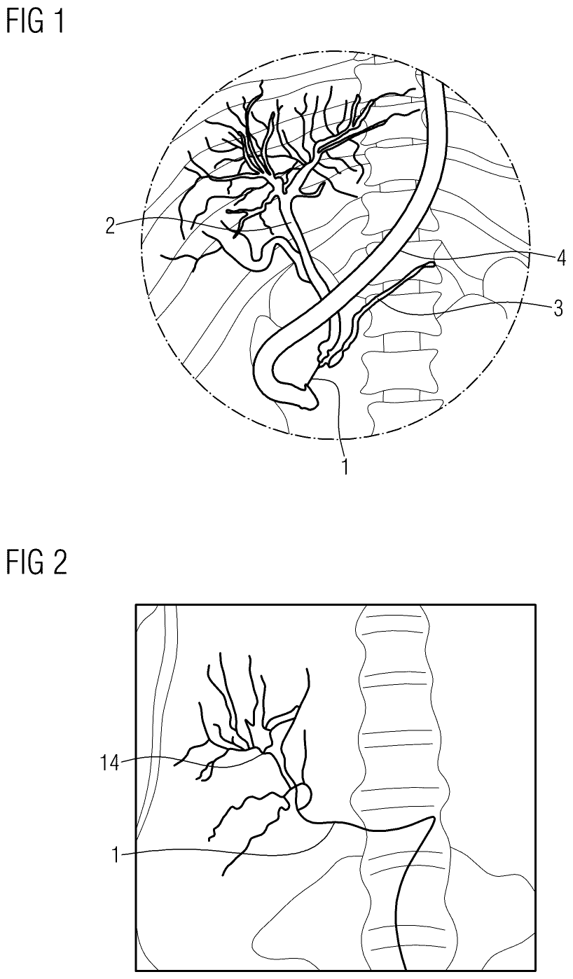

FIG. 1 shows an X-ray image of a human body in the region of the liver or bile ducts. A medical instrument 1, e.g., a guidewire or another catheter-like instrument with which the bile ducts may be cannulated or examined by penetration, may be identified on the X-ray image. The guidewire may be inserted into the bile ducts by a duodenoscope 4. Bile ducts 2 that have been visualized with a contrast medium may be identified in the background. For this purpose, for example, a guidewire was inserted into the bile duct 2 (e.g., the common bile duct) as a medical instrument 1 and the contrast medium was released into the bile duct 2 at the tip of the guidewire. In addition to the bile ducts 2, the X-ray image also shows the pancreatic channel 3 or pancreatic duct.

FIG. 2 shows an X-ray recording of an endovascular liver intervention, wherein the hepatic arteries 14 have likewise been filled with contrast medium. Here, the medical instrument 1 is an endovascular catheter.

Because the pancreas may react very sensitively to contrast media, (for example, during ERCP), the aim is to reduce the contrast medium administration or contrast medium administrations to a minimum and still be able to identify where the tip of the medical instrument is located, in particular in which vessel and possibly also at which point in the vessel. Contrast medium administration may also be minimized in the case of arterial interventions because the contrast medium has to be excreted via the kidneys and this may be very stressful for patients with impaired renal function.

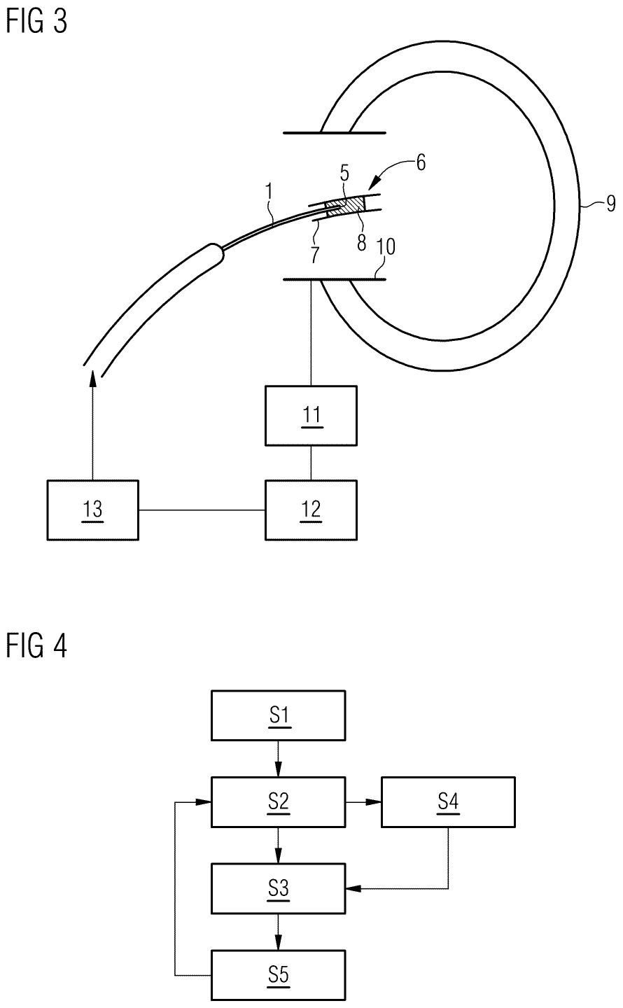

The disclosure is based on the idea of reducing or stopping contrast medium administration as quickly as possible when the contrast medium is detected in the respective vessel or channel. The system shown schematically in FIG. 3, for example, is provided for this purpose. The system includes a medical instrument 1, which may be a guidewire, a sphincterotome, or a catheter. The tip or the distal end 5 or the outlet of the medical instrument 1 is located in a surrounding region 6, which may represent the inside of a vessel or channel 7. A contrast medium 8 is injected into the surrounding region 6 via the distal end 5 or a distal end region of the medical instrument 1. In the present example, part of the channel 7 is filled with the contrast medium 8. In particular, a contrast medium plume forms at the outlet or distal end 5 of the medical instrument 1. Here, the contrast medium plume is already so large that it is limited by the walls of the channel 7. However, in principle, the contrast medium plume may also be smaller so that it is not limited by any channel walls. Of course, the contrast medium plume may also be larger and fill the entire channel 7.

The medical instrument, catheter, or guidewire may have a fluid channel for delivering the contrast medium through which the contrast medium may be injected via an opening near the distal end of the guidewire. The opening is located at the distal end and, in certain examples, directly at the distal end.

The tip or the outlet 5 of the medical instrument 1 is monitored by an imaging modality 9. The imaging modality 9 may be an X-ray system, such as a C-arm. The detector 10 of the imaging modality 9 of the system supplies image signals to a data/image processing unit 11. This image processing unit 11 is able to process an image of the imaging modality 9 and automatically detect the contrast medium 8 or a corresponding contrast medium plume on the image.

The image processing unit 11 sends a detection signal formed according to the detection to a control facility 12. In addition to a control functionality, the control facility 12 may also have a regulation functionality with feedback. In addition, the control facility 12 may also possibly have a data processing unit (for example, with a neural network) in order to learn target control values for a pumping facility 13. In one example, the control facility 12 generates a stop signal for the pumping facility 13 directly from the detection signal of the image processing unit 11 or also another control signal, for example, to reduce the speed of the contrast medium pump 13.

The pumping facility or contrast medium pump 13 may therefore be controlled by the control facility 12 via an interface. Optionally, there are measuring units in the contrast medium pump 13 and/or in the medical instrument 1 and/or at another location for detecting an amount of fluid that has flowed through and/or a flow rate and/or a pressure. These parameters may be used to regulate the contrast medium pump 13. The contrast medium pump 13 accordingly delivers the contrast medium into the medical instrument 1 where the contrast medium may be pumped through a fluid channel to the distal end 5 at or in the vicinity of which the contrast medium may escape into the surrounding region 6. The pumping facility or contrast medium pump may also have a mixing unit that may variably adjust a mixing ratio between contrast medium and, for example, saline solution. This enables a concentration of the contrast medium to be varied over time.

FIG. 4 is a schematic depiction of a block diagram of an exemplary embodiment of a method for detecting a distal end 5 of a medical instrument 1 in a surrounding region 6. Optionally, a reference image may be recorded in advance according to act S1. The reference image is recorded before the contrast medium 8 is introduced into the surrounding region 6 or the channel 7. The reference image may serve as a basis for image processing.

In act S2, the contrast medium 8 is ejected at the distal end 5 of the medical instrument 1 into the surrounding region 6.

In act S3, the ejected contrast medium 8 is automatically detected with an imaging modality 9 in the surrounding region 6. Therefore, the detection includes the actual identification of the contrast medium as such. The detection may also include detecting the shape of a propagation region of the contrast medium 8.

Optionally, an image refresh rate of the imaging modality 9 and/or its radiation dose may be increased according to act S4 as soon as the contrast medium is ejected S2. This enables image processing to be performed more precisely.

Finally, in act S4, the pumping facility 13 is actuated as a result of which the ejection of the contrast medium is varied according to act S2. In particular, the pumping facility 13 may be stopped or slowed down immediately in this act S5.

According to one embodiment, an adaptive contrast medium injection may take place for the smallest possible visible/usable amount. For this purpose, a contrast medium injection may be started by an input by the physician with the aid of the contrast medium pump 13. When the injection has started, the frame rate and/or dose per image of the X-ray device may be increased in order to achieve increased detection sensitivity. Optionally, a reference image may be recorded before/at the start of the injection for later subtraction.

As soon as a contrast medium leak is detected in the vicinity of the opening of the guidewire, the injection may be stopped as quickly as possible via an interface to the pump. Alternatively, the injection may be stopped as soon as sufficient vascular information may be derived. This may be the case as soon as the contrast medium enables a certain direction of the vessel to be identified.

Optionally, the pump parameters for a contrast medium injection may also be selected adaptively or learning-based. The amount of contrast medium for the required image signal (for example, image identification successful or predetermined quality is achieved) may be evaluated from previous injections. A new contrast medium injection may then be preset or carried out with parameters calculated on this basis.

In another embodiment, a section of a channel or vessel is digitally assembled. Such a digital assembly of a vessel or section of a vessel may be made from tiny, detected contrast medium distributions of the injections at different points in time. In each case, a tiny section of a vessel and, e.g., a local direction/extension of the vessel may be seen. A larger section of a vessel may be derived from this, optionally using motion compensation. The contrast medium measurements may be used for the registration of pre-interventional image data sets or segmentations thereof.

Advantageously, the above examples of a system or method make it easier to perform, for example, papillary cannulation during ERCP without significantly increasing the risks of pancreatitis due to the injected contrast medium.

It is to be understood that the elements and features recited in the appended claims may be combined in different ways to produce new claims that likewise fall within the scope of the present disclosure. Thus, whereas the dependent claims appended below depend on only a single independent or dependent claim, it is to be understood that these dependent claims may, alternatively, be made to depend in the alternative from any preceding or following claim, whether independent or dependent, and that such new combinations are to be understood as forming a part of the present specification.

While the present disclosure has been described above by reference to various embodiments, it may be understood that many changes and modifications may be made to the described embodiments. It is therefore intended that the foregoing description be regarded as illustrative rather than limiting, and that it be understood that all equivalents and/or combinations of embodiments are intended to be included in this description.

1. A method for detecting a contrast medium in a region surrounding an outlet of a medical instrument, the method comprising:
ejecting a contrast medium at a distal end of the medical instrument into the surrounding region;
automatically detecting the ejected contrast medium with an imaging modality in the surrounding region; and
automatically reducing or terminating the ejection of the contrast medium depending on a result of the detection.

2. The method of claim 1, wherein, during the ejecting of the contrast medium, at least one parameter of the contrast medium is detected and controlled or regulated depending on the result of the detection, and
wherein the at least one parameter of the contrast medium comprises an amount of fluid ejected, a flow rate, a pressure, a mixing ratio, a concentration, or a combination thereof.

3. The method of claim 2, further comprising, on a start of the ejecting of the contrast medium:
increasing a frame rate and/or a radiation dose of the imaging modality.

4. The method of claim 3, further comprising, before or at the start of the ejecting of the contrast medium:
recording a reference recording of the distal end of the medical instrument by the imaging modality,
wherein the reference recording is used in the detecting of the ejected contrast medium.

5. The method of claim 1, further comprising, on a start of the ejecting of the contrast medium:
increasing a frame rate and/or a radiation dose of the imaging modality.

6. The method of claim 1, further comprising, before or at a start of the ejecting of the contrast medium:
recording a reference recording of the distal end of the medical instrument by the imaging modality,
wherein the reference recording is used in the detecting of the ejected contrast medium.

7. The method of claim 1, wherein a pump for the contrast medium is slowed down or stopped to reduce or terminate the ejection of the contrast medium.

8. The method of claim 1, wherein, during the detecting of the contrast medium, a propagation region of the contrast medium is detected.

9. The method of claim 1, wherein the detecting of the contrast medium is based on automatic image identification and the result of the detection represents whether or not a predetermined image or a predetermined image content is identified.

10. The method of claim 1, further comprising:
repeating the ejecting, the detecting, and the reducing or the terminating,
wherein on each repetition, a position of the distal end of the medical instrument is changed and thus respective contrast medium administrations into different surrounding regions take place and images of the respective contrast medium administrations are superimposed.

11. The method of claim 1, wherein, for the ejecting of the contrast medium, one or more pump parameters are learned automatically in such a way that the detecting takes place according to a predetermined detection result.

12. A system comprising:
a medical instrument having an outlet for ejecting a predetermined contrast medium;
an imaging modality configured to detect a portion of the predetermined contrast medium ejected at the outlet of the medical instrument; and
a control facility configured to reduce or terminate an ejection of the predetermined contrast medium depending on a result of the detection.

13. The system of claim 12, wherein the medical instrument comprises a catheter, a sphincterotome, or a guidewire.

14. The system of claim 12, wherein the imaging modality comprises an X-ray apparatus, a magnetic resonance imaging (MRI) apparatus, or a sonography apparatus.

15. A computer program comprising instructions that, when executed by a system having a medical instrument, imaging modality, and control facility, cause the system to:
eject a contrast medium at a distal end of the medical instrument into a region surrounding the distal end of the medical instrument;
automatically detect the ejected contrast medium with the imaging modality in the surrounding region; and
automatically reduce or terminate the ejection of the contrast medium by the control facility depending on a result of the detection.
